(19) United States
(12) Patent Application Publication
(10) Pub. No.: US 2008/0270178 A1
(43) Pub. Date: Oct. 30, 2008

(54) Inventory Management System For A Medical Service Provider

(75) Inventors: Kristen McRae, Tucson, AZ (US); John M. Jackson, San Francisco, CA (US); Jennie L. Smith, San Francisco, CA (US); Bret Brodowy, Moraga, CA (US); Regina Leung, Berkeley, CA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: McKesson Specialty Distribution LLC

(21) Appl. No.: 11/756,486

(22) Filed: May 31, 2007

Related U.S. Application Data
(60) Provisional application No. 60/914,914, filed on Apr. 30, 2007.

(51) Int. Cl. G06Q 50/00

(52) U.S. Cl. 705/2; 707/104.1

(57) ABSTRACT

A system, method, and computer program product are provided for an improved inventory management system. The system supports a medical service provider by interfacing an inventory management application with a database of electronic medical records. In one embodiment, a single graphical user interface interfaces the inventory management application with the database of electronic medical records, thus providing a simplified, transparent solution as compared to the separate, independent, and non-interfaced systems currently available. By interfacing the inventory management application with the database of electronic medical records, inventory accuracy may be increased and processing times may be decreased.

### CROSS-REFERENCE TO RELATED APPLICATIONS

This application claims priority from U.S. Provisional Application No. 60/914,914, filed Apr. 30, 2007, which is hereby incorporated herein in its entirety by reference.

### FIELD OF THE INVENTION

In general, exemplary embodiments of the present invention relate to medication inventory management systems, and in particular to an inventory management system for a medical service provider.

### BACKGROUND OF THE INVENTION

Media sources often report on the rising costs of healthcare. With an aging baby-boomer generation, the costs of healthcare are expected to rise even higher. It has been reported that per capita health spending increased 39 percent between 1999 and 2003 for privately insured people, while worker's average hourly earnings grew only 14 percent. Some employers have reacted by beginning to eliminate healthcare benefits completely or shift more of the costs to employees. One of the most costly categories of healthcare is oncology healthcare. Some reports estimate that oncology costs exceed $200 billion annually. As a result, oncology service providers are constantly searching for ways to reduce healthcare costs while maintaining their level of patient care.

In addition to the problem of rising healthcare costs, many healthcare procedures still rely on dated information systems and paper-based records. Paper-based records are often susceptible to being lost, misfiled, damaged, or destroyed, leaving no record of the information contained therein. It has been reported that approximately 20 percent of medical tests require re-ordering simply because previous results were not available. Utilizing the latest and most accurate information is critical in providing optimal healthcare for patients. Yet current procedures and practices do not adequately rely on such information.

A large part of the daily operation of an oncology service provider involves the management of oncology medications. Using conventional systems, this often includes accessing paper patient records, manually tracking the administration of the medications, manually generating the cost of administration, and manually generating a patient bill relating to the administration of the medications. When a particular medication falls below a certain inventory level, additional quantities of the medication may be ordered by manually processing an order. Accurately tracking oncology medications is especially advantageous for an oncology service provider since oncology medications are very expensive relative to other traditional medications. As such, an oncology service provider should generally insure that sufficient medications are on hand to properly service its patients without incurring the cost associated with having an overabundance of costly medications in inventory.

Recent alternatives to the conventional oncology inventory management solution include cabinet-based inventory management systems, such as the Pyxis® OncologyStation™ system provided by Cardinal Health. However, because medications are contained within a cabinet, these systems require a great deal of physical space within the facility of an oncology service provider. Additionally, these systems do not generally provide interfaced solutions. If electronic medical records are desired, they require a separate and independent electronic medical records database. However, not all oncology service providers have the physical space or funds to dedicate to such systems. Additionally, in order to streamline oncology practice efficiencies and medication inventory management processes, further lowering costs, many oncology service providers desire interfacing with an electronic medical records system. By interfacing an electronic medical record application, database, and/or system with an inventory management application, inventory management processes may be improved by increasing inventory accuracy and decreasing processing times. Additionally, an interfaced system provides a simplified transparent solution as compared to the separate, independent, and non-interfaced systems currently available.

As a result, there is a need for a system, method, and computer program product that provides an improved oncology inventory management application such as an application that is interfaced with a database or system of electronic medical records. The inventory management system should be configured to process medication data, and to provide integration with the electronic medical records, thus improving the accuracy and decreasing the processing times associated with inventory management processes. The system may also be configured to interface with a transaction services hub, which may process medication charges associated with the administration of medications from the inventory.

### BRIEF SUMMARY OF THE INVENTION

Exemplary embodiments of the present invention provide an improvement over the known prior art by, among other things, providing a system for managing an inventory of medications for a medical service provider such as an oncology service provider. According to exemplary embodiments, the system reads data associated with medications, processes the data, and interfaces with an electronic medical records database, system, and/or application (hereinafter referred to collectively as a “database of electronic medical records”) to manage the inventory of medications. A method and computer program product for managing an inventory of medications for an oncology service provider are also provided.

In one embodiment, the inventory management system comprises a data input device configured to read medication data associated with the medications, a database of electronic medical records, and a processor configured to execute an inventory management application, wherein the inventory management application is configured to process the medication data and to interface with the database of electronic medical records for updating the inventory of medications. The inventory management application may be further configured to maintain a history of medications removed from the inventory for a patient and to provide a list of recent medications removed from the inventory based at least in part on the history of medication removed from the inventory for the patient, for use in selecting a current medication to be removed from the inventory for the patient. The inventory management application may also be configured to interface with the database of electronic medical records to access at least one of a diagnosis, stage, or regimen, and to project patient medication needs based at least in part on the diagnosis, stage, or regimen. The inventory management application may be further configured to automatically order medications based at least in part on the projected patient medication needs.

The data input device of the system may comprise at least one of a bar code scanner or an RFID reader. The database of electronic medical records may comprise an oncology-specific database or the medical service provider may comprise an oncology medical service provider. The inventory management application may comprise a web-based application and may be configured to allow automatic or manual ordering of medications. At least a portion of the database of electronic medical records may be hosted externally from the medical service provider. A single graphical user interface may be configured to interface with both the inventory management application and the database of electronic medical records so that from a user's perspective, the inventory management application and the database of electronic medical records appear integrated. At least one medication in the inventory of medications may be received in a predetermined concentration, wherein the database of electronic medical records may include a dosage of the medication to be administered to a patient, and wherein the inventory management application may automatically calculate a draw-up amount of the medication based at least in part on the predetermined medication concentration and the dosage of medication to be administered to the patient. The data input device may be configured to communicate with the inventory management application to verify that medication leaving the inventory of medications for administration to a patient is the proper medication. The system may be configured to interface with an existing transaction services hub for processing medication charges associated with medications leaving the inventory for administration to a patient. At least a portion of the inventory of medications may be received in full medication vials, and the inventory management application may be configured to track inventory levels associated with the medications in partial vial increments.

As a result, embodiments of the present invention provide a system, method, and computer program product that provide an improved oncology inventory management system. The system supports a medical service provider, such as an oncology service provider, by interfacing an inventory management application with a database of electronic medical records. By interfacing the inventory management application with the database of electronic medical records, inventory accuracy may be increased and processing times may be decreased.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a diagram illustrating an inventory management system of an exemplary embodiment of the present invention;

FIG. 2 is a diagram illustrating an inventory management system in accordance with another exemplary embodiment of the present invention; and

FIG. 3 is a block diagram of an exemplary electronic device configured to manage an inventory of medications for an oncology service provider of exemplary embodiments of the present invention.

### DETAILED DESCRIPTION OF THE INVENTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

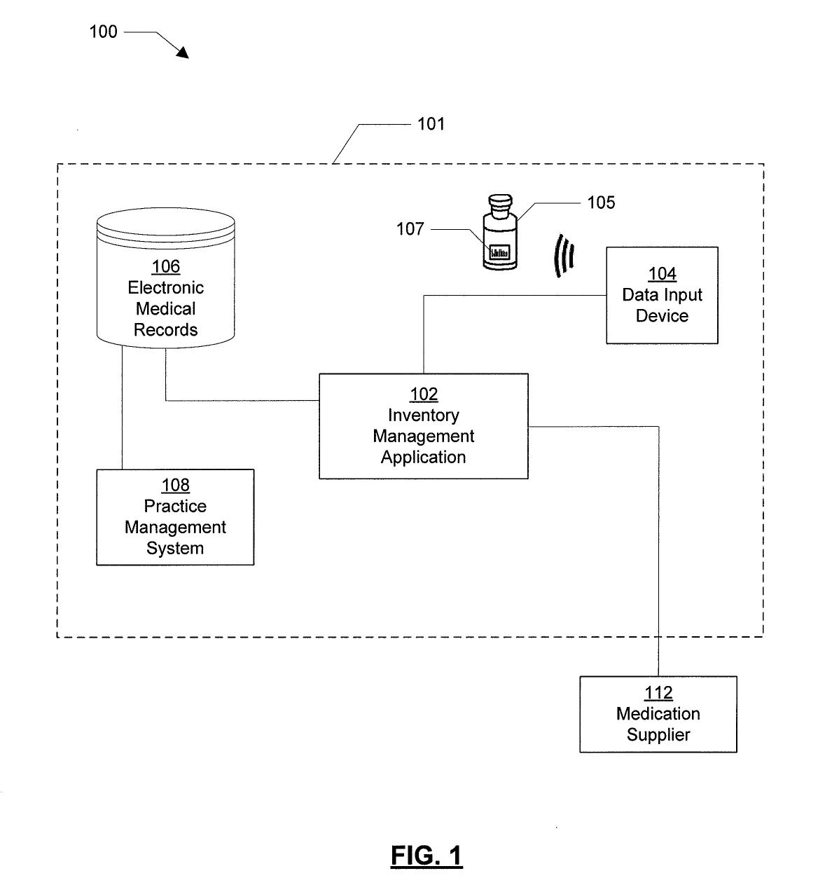

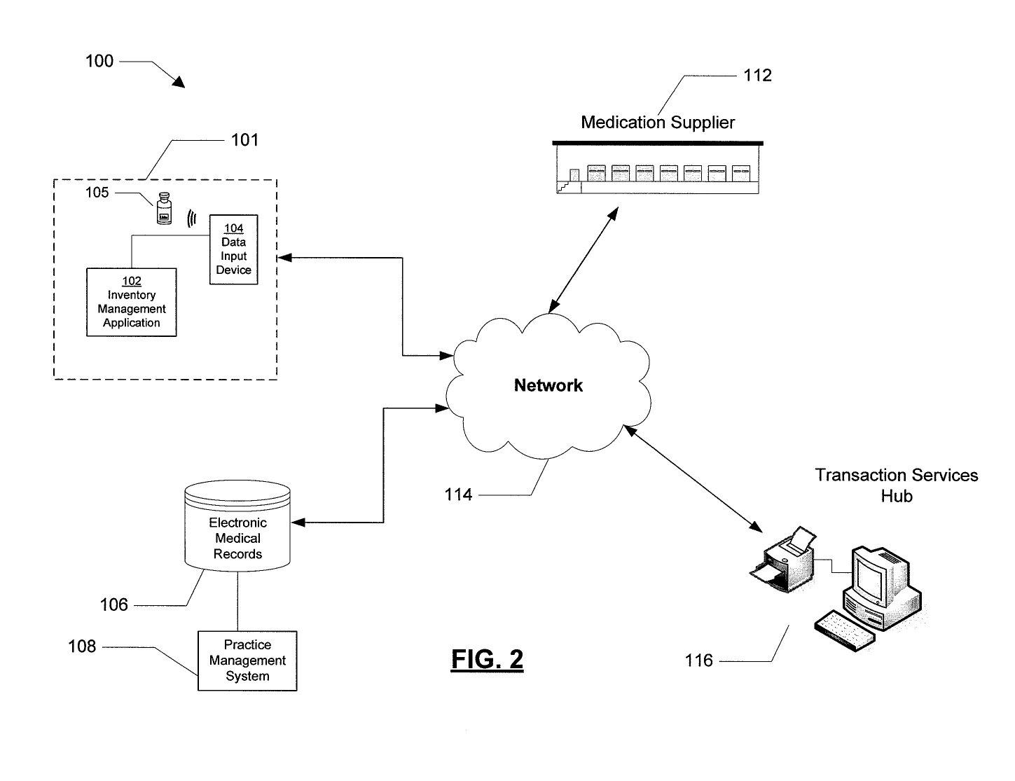

An embodiment of the present invention may be better understood with reference to FIG. 1, which is a diagram illustrating an inventory management system 100 for managing an inventory of medications for an oncology service provider 101, and FIG. 2, which is another embodiment of an inventory management system 100. It should be noted that although the depicted embodiments relate to an improved inventory management system for an oncology service provider, embodiments of the present invention may be advantageous for any medical service provider and thus should not be limited to an oncology service provider. The system, method and computer program product of embodiments of the present invention manage the inventory of an oncology service provider 101 by providing an inventory management application 102 that processes medication data relating to medications entering and leaving an inventory of medications and interfaces with a database of electronic medical records. The inventory management application 102 may interface with the database of electronic medical records 106 in a variety of ways as are known in the art. In some embodiments, a single graphical user interface is provided that interfaces the inventory management application 102 with the database of electronic medical records 106 so that from a user's perspective, the inventory management application 102 and the database of electronic medical records appear integrated. It should be noted that in such embodiments, the graphical user interface may be a separate application, or may be associated with either or both the inventory management application 102 and the database of electronic medical records 106. Additionally, the graphical user interface may provide single sign-on capability.

Medications entering the inventory of medications generally relate to medications received from a medication supplier 112, and medications leaving the inventory of medications typically relate to medications that are removed or withdrawn from the inventory prior to being administered to patients. Exemplary embodiments of the present invention manage the inventory of oncology medications by processing medication data relating to medications entering and leaving the inventory, and interfacing with a database of electronic medical records, thus creating an improved inventory management tool. Unlike the cabinet-based inventory management systems described above, where medications must be stored within the system itself (thus occupying a great deal of physical storage space within the facility of the oncology service provider), exemplary embodiments of the present invention do not require medications to be stored within a cabinet of the system. Rather, various embodiments of the present invention allow the oncology service provider 101 to use its current storage system. As a result, various embodiments of the present invention do not present the same physical space and economic constraints presented by current cabinet-based systems.

As shown in FIG. 1, the inventory management system 100 of an exemplary embodiment includes an inventory management application 102 and a data input device 104 in communication with the inventory management application 102. As will be discussed in more detail below, the inventory management application 102 interfaces with a database of electronic medical records 106. As shown in the depicted embodiment, the database of electronic medical records 106 may communicate with a practice management system 108 that manages patient check in/out, patient scheduling, and/or patient billing. In various embodiments, the practice management system 108 may be a separate system, or may be part of the database of electronic medical records 106. The inventory management application 102 also communicates with a medication supplier 112. Although the medication supplier 112 may be any medication supplier capable of communicating with the inventory management application 102, in the exemplary embodiment, the medication supplier 112 is an online medication supply system, such as the McKessson® SAP® system. The data input device 104 in the exemplary embodiment is a barcode scanner used to scan barcode labels 107 that provide medication data associated with the medications 105. In other embodiments, the data input device 104 may comprise other devices capable of reading information associated with the medications 105, including, but not limited to, Radio Frequency Identification (RFID) readers configured to read RFID transponders associated with the medications. As will be discussed in more detail below, the medication data may be read by the data input device 104 as medications 105 are removed from inventory, such as when medications are administered to patients. Medication data may also be read when new medications 105 are added to the inventory. The medication data includes information relating to the medications 105, such as the medication name, dose strength, package size, and an identification number. In various embodiments, this information may also include the medication quantity. In other embodiments this data may comprise additional information including, but is not limited to, the medication manufacturer, the lot number, any expiration date information, and the like. In the exemplary embodiment, the inventory management application 102 is configured to not only receive medication data from the data input device, but can also accept manual input of medication data, such as the manual input of the medication quantity, via a computer interface, such as a keyboard, touchscreen or the like.

The inventory management application 102 of the exemplary embodiment is a web-based software application that may be hosted by an external mainframe (not shown) accessible by one or more clients. Although in other embodiments, the inventory management application 102 need not be web-based and may a local application located at the medical service provider's 101 site. The exemplary embodiment of the inventory management application is advantageous in that it may not require on-site installation and maintenance, and thus may minimize disruptions to an oncology service provider 101 for product upgrades and other software maintenance issues.

With regard to medications leaving the inventory of medications for the oncology service provider, the inventory management application 102 of embodiments of the present invention provides an electronic list of medications available in the inventory of medications and allows a user to select a particular medication from the inventory for administration to a patient. The data input device 104 may be used to scan or read the label 107 of the medication 105 as the medication 105 is physically removed from the inventory prior to being administered to the patient. As a result, the inventory management application 102 may verify that the medication 105 leaving the inventory of medications for administration to the patient is the proper medication.

As noted above, the inventory management application 102 interfaces with a database of electronic medical records 106. In the exemplary embodiment, the database of electronic medical records 106 is stored in a memory device and hosted by the oncology service provider 101 and comprises an oncology-specific system of electronic medical records that contains electronic medical records associated with patients of the oncology service provider 101. It should be noted however, in other embodiments the database of electronic medical records 106 may comprise a database that includes patients of various medical service providers. The electronic medical records include clinical data associated with each patient (patient clinical data). In various embodiments the patient clinical data may include, but is not limited to, patient demographics, clinical diagnoses, oncology stages, treatment regimens, and medication usage history. For example, in the exemplary embodiment, the patient clinical data includes patient ICD (International Statistical Classification of Diseases and Related Health Problems) codes including primary, secondary, and/or tertiary data; oncology stages, patient medication regimens and associated CPT (Current Procedural Terminology) codes; medication dose amount specific to the patient; medication dose form and strength; and other pertinent clinical information, such as complete blood count and vital information. Although the database of electronic medical records 106 in the exemplary embodiment is associated with the patients of the oncology service provider 101, in other embodiments, the database of electronic medical records 106 may be part of a large aggregate database of electronic medical records.

By interfacing with the database of electronic medical records 106 associated with patients of the oncology service provider 101, data relating to the administration of medications may be accessed and monitored as medications 105 are administered to patients. Although the database of electronic medical records 106 of the exemplary embodiment is hosted by the oncology service provider 101, in other embodiments, such as the one depicted in FIG. 2, at least a portion of the database of electronic medical records 106 may be hosted externally. Additionally, although the inventory management application 102 may interface with the database of electronic medical records 106 in any manner known in the art, in the exemplary embodiment, the inventory management application 102 interfaces with the database of electronic medical records 106 through a single graphical user interface as described above. As such, in various embodiments a user may sign onto either the inventory management application 102 or the database of electronic medical records 106 to access the functionality and data of either. In other embodiments, the inventory management application 102 may also be configured to import patient data from external sources. The inventory management application may do this in any manner known in the art.

The inventory management application 102 interfaces with the database of electronic medical records 106 to improve inventory management processes by increasing inventory accuracy and decreasing processing times. For example, as noted above, prior to physically removing a medication 105 from the inventory of medications for administration to a patient, a user may be provided with a list of medications from which the user selects the medication to be removed. In one embodiment of the present invention, a master list of medications is provided to the user. The master list of medications represents most, if not all, of the medications available in the inventory. However, in many instances, the master list of medications includes a vast number of medications, many of which are not relevant to a particular patient's medication regimen. Therefore, in another embodiment of the present invention, the inventory management application 102 streamlines this process and is configured to maintain a history of the medications removed from the inventory for a particular patient. As such, when the time comes for a user to select from the inventory management application a current medication to be removed from the inventory for administration to a patient, the inventory management application 102 may provide the user with an abbreviated list of medications. The abbreviated list of medications may represent those medications that were most recently removed for that patient. In various embodiments, the inventory management application 102 may allow the oncology service provider 101 to specify a period of time for which to maintain the history. This period may be the same for all patients of the oncology service provider, or may be patient-specific.

Embodiments of the present invention may also improve inventory management processes by interfacing with a database of electronic medical records 106 to project patient medication needs. In various embodiments of the present invention, the database of electronic medical records 106 may include oncology diagnoses, stages, and/or treatment regimens. The diagnoses, stages, and/or treatment regimens may be patient-specific or may relate to general diagnoses, stages, and/or treatment regimens. In the depicted embodiment, diagnoses may relate, but are not limited to, the identification of one or more cancers. Stages may relate, but are not limited to, an extent of progress of one or more cancers. Treatment regimens may include, but are not limited to, treatment plans, which often include the administration of one or more medications, as well as other courses of treatments, such as diets and/or exercise. By interfacing with the database of electronic medical records 106, the inventory management application 102 of one embodiment of the present invention may project patient medication needs based on one or more of a patient's diagnosis, stage, or regimen. Notably, the patient medication needs may, but need not, be determined before medication is removed from the inventory. As a result, the inventory management application 102 may project medications and amounts (including dosages, etc.) that will be needed for administration to a patient. The inventory management application 102 may also update and/or establish order quantities for the oncology service provider 101 based, at least in part, on this information, and through communication with the medication supplier 112, the inventory management application 102 may automatically (i.e., with manual intervention) order one or more of the medications. It should be noted that in various embodiments, the system 100 is configured to allow other medications to be ordered according to their usage by the oncology service provider, which may be tracked by the inventory management application 102. Additionally, the system 100 is also configured to permit manual ordering of one or more medications.

Embodiments of the present invention may also improve inventory management processes and accuracy by performing functions that, under many existing inventory management systems, must be manually performed by a user, such as a person administering medications to a patient. For instance, many medications, such as those that are administered in liquid form, are received by oncology service providers in predetermined concentrations. However, dosages to be administered to patients do not always match the concentrations of the medications in the inventory. To accommodate the administration dosage, those who administer the medications typically manually calculate the amount of medication to “draw-up” into a syringe that contains an additional and innocuous substance so that the proper concentration of the medication is contained in the dosage. The inventory management application in accordance with various embodiments of the present invention automatically calculates the draw-up amount (i.e., the amount of medication at the predetermined concentration that should be drawn into in the syringe). As a result, this reduces manual processing time and increases both the accuracy of the administration of the medication, as well as the accuracy of the inventory.

Embodiments of the present invention may also improve the inventory processes and accuracy by tracking medications in smaller, more accurate increments than current inventory management systems. For instance, many medications are received in full medication containers that contain a predetermined amount of medication. Examples include medications that are administered in liquid form and that are received in vials that contain a predetermined volume of medication. Often, however, the prescribed dosage of the medication may require less than a full vial's worth of the medication. After administering a portion of the vial to the patient, existing inventory management systems would consider the entire vial of medication to have left the inventory. Unfortunately, this leads to an overage of medication as compared to the inventory count. On the other hand, if the partial vial is removed from the inventory and discarded, this amounts to wastage of medication. In either event, the inventory management application 102 of one embodiment of the present invention may improve this situation by being configured to track inventory levels for medications in partial increments, the level of which may be determined by the oncology medical service provider 101. For example, a full vial of a medication may comprise 10 milliliters (ml) of medication. If only 7 ml are used for administration to a patient, the inventory management application 102 may be configured to track this medication in milliliter increments such that the inventory count for this medication would indicate that 3 ml's remain in the inventory. This advantage also translates to more accurate patient billing because the patient is more likely to be billed according to the specific amount of medication that was administered, as opposed to the amount of medication that was said to have left the inventory, in this case 7 ml vs. one full vial.

Under existing systems, medical service providers must have separate practice management system applications that manage patient check in/out, patient scheduling, and patient billing. In some embodiments of the present invention, the inventory management application 102 may also interface with an external system that processes patient billing for various medical service providers. Additionally, another system may handle the patient check in/out and scheduling functions, thus alleviating the need for additional practice management system. FIG. 2 represents such an embodiment. In the depicted embodiment, the oncology service provider 101 may communication over a network 114 with a transaction service hub 116 that handles billing services for the oncology medical service provider 101. It should be noted that although only one medical service provider is shown, there may be many medical service providers whose billing services are handled by the transaction services hub 116 and who may communicate over the network 114. In various embodiments, the network 114 may be a LAN, a WAN, the Internet, etc. As also shown in depicted embodiment, the inventory management application 102 may interface with a database of electronic medical records 106 by communicating with the database 106 over the network 114. Additionally, the inventory management application 102 may communicate over the network 114 with a medication supplier 112 for ordering medications. In this manner, several medical service providers may share a single database of electronic medical records and may communication with one or more medication suppliers over the network 114.

The inventory management system may also include a printer (not shown) that may be configured to print out certain output. The output may include, for example, labels that are generated upon medication dispensation. The labels may include information relating to the dispensation of the medication including, for example, identifying information such as facility, physician, and patient names, patient identification numbers, and medication dispensation information such as medication name, dose amount, dose strength, and NDC (National Drug Code) numbers. Output may also include certain inventory and cost reports. The reports may include, for example, activity summaries relating to medication dispensation events; medication utilization summaries; inventory count summaries; inventory cost summaries; and medication regimen costs per patient; and other summaries relating to data accessible by the inventory management application.

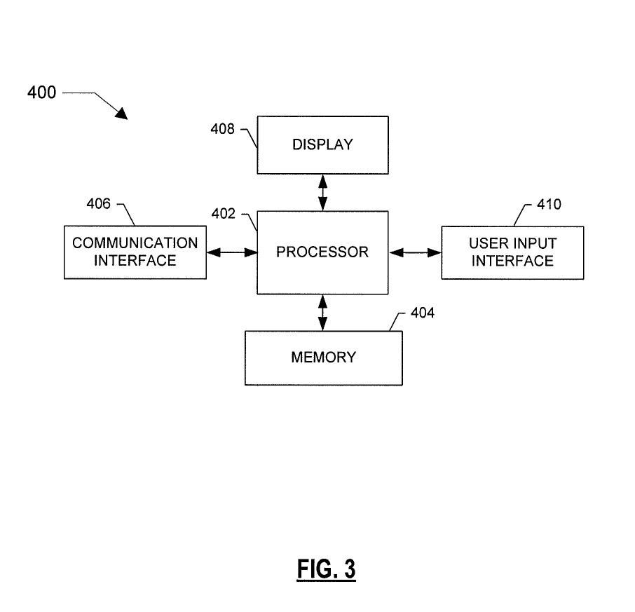

The foregoing merely illustrates how exemplary embodiments of the present invention manage an inventory of medications for an oncology service provider. Referring now to FIG. 3, a block diagram of an exemplary electronic device 400 (e.g., mainframe, PC, laptop, PDA, etc.) configured to execute the method and computer program product for managing an inventory of medications for an oncology service provider of exemplary embodiments of the present invention is shown. The electronic device may include various means for performing one or more functions in accordance with exemplary embodiments of the present invention, including those more particularly shown and described herein. It should be understood, however, that the electronic device may include alternative means for performing one or more like functions, without departing from the spirit and scope of the present invention. As shown, the electronic device may generally include means, such as a processor, controller, or the like 402 connected to a memory 404, for performing or controlling the various functions of the entity.

The memory can comprise volatile and/or non-volatile memory, and typically stores content, data or the like. For example, the memory typically stores content transmitted from, and/or received by, the electronic device. Also for example, the memory typically stores software applications, instructions or the like for the processor to perform steps associated with operation of the electronic device in accordance with embodiments of the present invention. In particular, the memory 404 may store computer program code for an application and other computer programs. For example, in one exemplary embodiment of the present invention, the memory may store computer program code for, among other things reading medication data associated with medications entering or leaving the inventory and interfacing with a database of electronic medical records to process the medication data, for updating the inventory of medications. In relation to the embodiment depicted in FIG. 1 and described above, the memory may include the inventory management application 102 and/or the database of electronic medical records 106 with the processor 402 configured to execute the inventory management application to perform as described above.

In addition to the memory 404, the processor 402 can also be connected to at least one interface or other means for displaying, transmitting and/or receiving data, content or the like. In this regard, the interface(s) can include at least one communication interface 406 or other means for transmitting and/or receiving data, content or the like. For example, the communication interface may provide for communications with an input device 104. The communication interface may also include at least one user interface that can include a display 408 and/or a user input interface 410. The user input interface, in turn, can comprise any of a number of devices allowing the electronic device to receive data from a user, such as a keypad, a touch display, a joystick or other input device.

As described above and as will be appreciated by one skilled in the art, embodiments of the present invention may be configured as a method and apparatus. Accordingly, embodiments of the present invention may be comprised of various means including entirely of hardware, entirely of software, or any combination of software and hardware. Furthermore, embodiments of the present invention may take the form of a computer program product consisting of a computer-readable storage medium (e.g., the memory 404 of FIG. 3) and computer-readable program instructions (e.g., computer software such as the computer software that comprises the inventory management application 102) stored in the storage medium. Any suitable computer-readable storage medium may be utilized including hard disks, CD-ROMs, optical storage devices, or magnetic storage devices.

Exemplary embodiments of the present invention have been described above with reference to block diagrams and flowchart illustrations of methods, apparatuses (i.e., systems) and computer program products. It will be understood that each block of the block diagrams and flowchart illustrations, and combinations of blocks in the block diagrams and flowchart illustrations, respectively, can be implemented by various means including computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

As a result, embodiments of the present invention provide a system, method, and computer program product for an improved oncology inventory management system. The system supports a medical service provider, such as an oncology service provider, by interfacing an inventory management application with a database of electronic medical records. By interfacing the inventory management application with the database of electronic medical records, inventory accuracy may be increased and processing times may be decreased.

Many modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An inventory management system for managing an inventory of medications for a medical service provider, said system comprising:
a data input device configured to read medication data associated with the medications;
a database of electronic medical records; and
a processor configured to execute an inventory management application,
wherein the inventory management application is configured upon execution by the processor to process the medication data and to interface with the database of electronic medical records for updating the inventory of medications.

2. The inventory management system of claim 1, wherein the inventory management application is further configured to maintain a history of medications removed from the inventory for a patient.

3. The inventory management system of claim 2, wherein the inventory management application is further configured to provide a list of recent medications removed from the inventory based at least in part on the history of medication removed from the inventory for the patient, for use in selecting a current medication to be removed from the inventory for the patient.

4. The inventory management system of claim 1, wherein the inventory management application is configured to interface with the database of electronic medical records to access at least one of the group consisting of a diagnosis, stage, or regimen, and to project patient medication needs based at least in part on the diagnosis, stage, or regimen.

5. The inventory management system of claim 4, wherein the inventory management application is further configured to automatically order medications based at least in part on the projected patient medication needs.

6. The inventory management system of claim 1, wherein the data input device comprises at least one of a bar code scanner or an RFID reader.

7. The inventory management system of claim 1, wherein the database of electronic medical records comprises an oncology-specific database of electronic medical records or the medical service provider is an oncology medical service provider.

8. The inventory management system of claim 1, wherein the inventory management application comprises a web-based application.

9. The inventory management system of claim 1, wherein the inventory management application is configured to allow automatic or manual ordering of medications.

10. The inventory management system of claim 1, wherein at least a portion of the database of electronic medical records is hosted externally from the medical service provider.

11. The inventory management system of claim 1, wherein a single graphical user interface is configured to provide interfacing between the inventory management application and the database of electronic medical records so that from a user's perspective, the inventory management application and the database of electronic medical records appear integrated.

12. The inventory management system of claim 1, wherein at least one medication in the inventory of medications is received in a predetermined concentration, wherein the database of electronic medical records includes a dosage of the medication to be administered to a patient, and wherein the inventory management application automatically calculates a draw-up amount of the medication based at least in part on the predetermined medication concentration and the dosage of medication to be administered to the patient.

13. The inventory management system of claim 1, wherein the data input device is configured to communicate with the inventory management application to verify that medication leaving the inventory of medications for administration to a patient is the proper medication.

14. The inventory management system of claim 1, wherein the system is configured to interface with an existing transaction services hub for processing medication charges associated with medications leaving the inventory for administration to a patient.

15. The inventory management system of claim 1, wherein at least a portion of the inventory of medications is received in full medication vials, and wherein the inventory management application is configured to track inventory levels associated with the medications in partial vial increments.

16. A method of managing an inventory of medications for a medical service provider, the method comprising:
reading medication data associated with medications entering or leaving the inventory using a data input device;
processing the medication data with an inventory management application; and
interfacing the inventory management application with a database of electronic records to update the inventory of medications.

17. The method of claim 16, further maintaining a history of medications removed from the inventory for a patient.

18. The method of claim 17, further comprising providing a list of recent medications removed from the inventory based at least in part on the history of medications removed from the inventory for the patient, for use in selecting a current medication to be removed from the inventory for the patient.

19. The method of claim 16, further comprising interfacing with the database of electronic medical records to access at least one of the group consisting of a diagnosis, stage, or regimen, and projecting patient medication needs based at least in part on the diagnosis, stage, or regimen.

20. The method of claim 19, further comprising automatically ordering medications based at least in part on the projected patient medication needs.

21. The method of claim 16, wherein reading medication data comprises reading at least one of bar code medication data or RFID medication data.

22. The method of claim 16, wherein interfacing with a database of electronic medical records comprises interfacing with a database of oncology-specific electronic medical records or the medical service provider is an oncology medical service provider.

23. The method of claim 16, wherein the inventory management application comprises a web-based application.

24. The method of claim 16, further comprising providing automatic or manual ordering of at least some medications identified to be leaving inventory.

25. The method of claim 16, wherein interfacing with a database of electronic records comprises interfacing with a database of electronic records, at least of portion of which is hosted externally from the medical service provider.

26. The method of claim 13, wherein a single graphical user interface is configured to provide interfacing between the inventory management application and the database of electronic medical records so that from a user's perspective, the inventory management application and the database of electronic medical records appear integrated.

27. The method of claim 13, further comprising receiving into the inventory at least one medication in a predetermined concentration, wherein the database of electronic medical records includes a dosage of the medication to be administered to a patient, and calculating a draw-up amount of the medication based at least in part on the predetermined medication concentration and the dosage of medication to be administered to the patient.

28. The method of claim 13, wherein the data input device is configured to communicate with the inventory management application to verify that medication leaving the inventory of medications for administration to a patient is the proper medication.

29. The method of claim 13, further comprising interfacing with an existing transaction services hub for processing medication charges associated with medications leaving the inventory for administration to a patient.

30. The method of claim 13, further comprising receiving at least a portion of the inventory of medications in full medication vials, and tracking inventory levels associated with the medications in partial vial increments.

31. A computer program product for managing an inventory of medications for a medical service provider, the computer program product comprising:
a first executable portion for processing medication data and interfacing with a database of electronic records to update the inventory of medications to account for the medications entering or leaving the inventory.

32. The computer program product of claim 31, further comprising a second executable portion for maintaining a history of medications removed from the inventory for a patient.

33. The computer program product of claim 32, further comprising a third executable portion for providing a list of recent medications removed from the inventory based at least in part on the history of medications removed from the inventory, for the patient for use in selecting a current medication to be removed from the inventory.

34. The computer program product of claim 31, further comprising a second executable portion for accessing at least one of the group consisting of a diagnosis, stage, or regimen, and a third executable portion for projecting patient medication needs based at least in part on the diagnosis, stage, or regimen.

35. The computer program product of claim 34, further comprising a fourth executable portion for automatically ordering medications based at least in part on the projected patient medication needs.

36. The computer program product of claim 31, wherein the medication data is provided by a data input device comprising at least one of a bar code scanner or an RFID reader.

37. The computer program product of claim 31, wherein the database of electronic medical records comprises an oncology-specific database of electronic medical records or the medical service provider is an oncology medical service provider.

38. The computer program product of claim 31, wherein the computer program product comprises a web-based application.

39. The computer program product of claim 31, wherein the first executable portion is configured to allow automatic or manual ordering of at least some medications identified to be leaving the inventory.

40. The computer program product of claim 31, wherein at least a portion of the database of electronic medical records is hosted externally from the medical service provider.

41. The computer program product of claim 31, wherein a single graphical user interface is configured to provide interfacing between the inventory management application and the database of electronic medical records so that from a user's perspective, the inventory management application and the database of electronic medical records appear integrated.

42. The computer program product of claim 31, further comprising receiving into the inventory at least one medication in a predetermined concentration, wherein the database of electronic medical records includes a dosage of the medication to be administered to a patient, and further comprising a second executable portion for calculating a draw-up amount of the medication based at least in part on the predetermined medication concentration and the dosage of medication to be administered to the patient.

43. The computer program product of claim 31, wherein at least a portion of the medication data is used to verify that medication leaving the inventory of medications for administration to a patient is the proper medication.

44. The computer program product of claim 31, further comprising a second executable portion for interfacing with an existing transaction services hub for processing medication charges associated with medications leaving the inventory for administration to a patient.

45. The computer program product of claim 31, wherein at least a portion of the inventory of medications are received in full medication vials, and further comprising a second executable portion for tracking inventory levels associated with the medications in partial vial increments.
